(19) United States
(12) Patent Application Publication
(10) Pub. No.: US 2024/0218122 A1
(43) Pub. Date: Jul. 4, 2024

(54) RADIOPAQUE HYDROGELS AND PRECURSORS THEREOF HAVING ENHANCED RADIOPACITY

(71) Applicant: Boston Scientific Scimed, Inc., Maple Grove, MN (US)

(72) Inventors: Yen-Hao Hsu, Shrewsbury, MA (US); Cristian Parisi, Boston, MA (US)

(73) Assignee: Boston Scientific Scimed, Inc., Maple Grove, MN (US)

(21) Appl. No.: 18/400,970

(22) Filed: Dec. 29, 2023

Related U.S. Application Data
(60) Provisional application No. 63/436,238, filed on Dec. 30, 2022.

(51) Int. Cl. C08G 65/26; A61K 51/00; C08J 3/02

(52) U.S. Cl. CPC C08G 65/2609; A61K 51/00; C08G 65/2624; C08J 3/02; C08G 2210/00; C08J 2371/02

(57) ABSTRACT

In some embodiments, the present disclosure pertains to multi-arm polymers having at least one polymer arm that comprises a reactive radiopaque end moiety. The radiopaque end moiety comprises a radiopaque dicarboxylic acid ester moiety containing one or more radiopaque atoms, wherein one of two ester groups of the radiopaque dicarboxylic acid ester moiety forms a link to the polymer arm, and wherein the other of the two ester groups of the radiopaque dicarboxylic acid ester moiety corresponds to a portion of a reactive electrophilic group. The present disclosure also pertains to methods of forming such multi-arm polymers and to hydrogels formed from such multi-arm polymers and a polyamino compound comprising a plurality of primary amine groups that form crosslinks with the multi-arm polymers. The present disclosure further pertains to methods and systems for forming such hydrogels and methods of treatment using such hydrogels.

### CROSS-REFERENCE TO RELATED APPLICATIONS

This application claims the benefit of U.S. Provisional Patent Application Ser. No. 63/436,238 filed on Dec. 30, 2022, the disclosure of which is incorporated herein by reference.

### FIELD

The present disclosure relates to radiopaque crosslinked hydrogels, to methods of making and using radiopaque crosslinked hydrogels and to precursors thereof, among other aspects. The radiopaque crosslinked hydrogels of the present disclosure are useful, for example, in various medical applications.

### BACKGROUND

In vivo crosslinked hydrogels based on star-poly(ethylene glycol) (star-PEG) polymers functionalized with reactive ester end groups which react with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR Clinical Trial Results,” Imaging Technology News, Oct. 27, 2016.

Hydrogels in which some of the star-PEG branches have been functionalized with 2,3,5-triiodobenzamide (TIB) groups replacing part of ester end groups, such as SpaceOAR® Vue, have also been developed to provide enhanced radiocontrast properties.

While the above approach is effectual, some issues arise as a result of incorporation of the functional group, TIB. First, in order to functionalize TIB on 8-arm PEG, the binding site from succinimidyl glutarate (SG) must be sacrificed for each functionalized arm causing longer gel times. Moreover, the entire functionalization process involves multiple steps, typically five steps, from commercially available hydroxyl-terminated 8-arm PEG to its functionalized form with two different end groups (TIB and SG groups). This complex process of synthesizing the 8-arm PEG results in a significant increase of the product cost, and more complex product quality control activities.

### SUMMARY

The present disclosure is directed to an alternative approach to that described above, in which a radiopaque anhydride precursor compound, preferably an iodinated cyclic anhydride compound, is used to incorporate radiopaque atoms into multi-arm polymers, followed by a further reaction with a reagent suitable for the creation of reactive functional groups as active end groups.

In some embodiments, the present disclosure pertains to multi-arm polymers having at least one (e.g., one, two, three, four, five, six, seven, eight or more) polymer arm that comprises a reactive radiopaque end moiety. The radiopaque end moiety comprises a radiopaque dicarboxylic acid ester moiety containing one or more radiopaque atoms, wherein one of two ester groups of the radiopaque dicarboxylic acid ester moiety forms a link to the polymer arm, and wherein the other of the two ester groups of the radiopaque dicarboxylic acid ester moiety corresponds to a portion of a reactive electrophilic group.

In some embodiments, the reactive electrophilic group is selected from an imidazole ester group, a benzotriazole ester group, and an imide ester group.

In some embodiments, which can be used in conjunction with any of the above embodiments, the one or more radiopaque atoms of the radiopaque dicarboxylic acid ester moiety are selected from Br, I, Bi, Ba, Gd, Ta, Zn, W and Au.

In some embodiments, which can be used in conjunction with any of the above embodiments, the radiopaque dicarboxylic acid ester moiety is an iodine-containing dicarboxylate ester moiety, for example, an iodine-containing phthalate ester moiety.

In some embodiments, which can be used in conjunction with any of the above embodiments, the multi-arm polymer further comprises an additional ester group disposed between the radiopaque dicarboxylic acid ester moiety and the polymer arm. For example, the additional ester group may be a C₂-C₈ alkanoate ester group.

In some embodiments, which can be used in conjunction with any of the above embodiments, the at least one polymer arm is selected from poly(alkylene oxide) arms, polyoxazoline arms, and poly(N-vinyl pyrrolidone) arms.

In some embodiments, which can be used in conjunction with any of the above embodiments, the at least one polymer arm extends from a core selected from a polyol residue core and a silsesquioxane core.

In some embodiments, the present disclosure pertains to systems for forming hydrogels, the systems comprising a multi-arm polymer in accordance with any of the above embodiments and a polyamino compound comprising a plurality of primary amine groups that form crosslinks with the multi-arm polymer.

In some embodiments, the polyamino compound comprises alkylamino groups that include the plurality of primary amine groups. For example, the alkylamino groups may be of the formula —(CH₂)ₓ—NH₂, where x is 1, 2, 3, 4, 5 or 6.

In some embodiments, which can be used in conjunction with any of the above embodiments, the systems comprise a first precursor composition that comprises the polyamino compound, a second precursor composition that comprises the multi-arm polymer, and an optional accelerant composition. In some embodiments, the first precursor composition may be provided in a syringe barrel, the second precursor composition may be provided in a vial, and the accelerant composition may be provided in a syringe barrel (e.g., different from the syringe barrel comprising the first precursor composition).

In some embodiments, which can be used in conjunction with any of the above embodiments, the systems further comprise a delivery device.

In some embodiments, the present disclosure is directed to medical hydrogels (e.g., hydrogels that are biocompatible, physiologically acceptable, bioresorbable, and/or biodegradable) that are formed by crosslinking a multi-arm polymer in accordance with any of the above embodiments and a polyamino compound in accordance with any of the above embodiments.

In some embodiments, the present disclosure is directed to methods of treatment comprising administering to a subject a mixture that comprises a multi-arm polymer in accordance with any of the above embodiments and a polyamino compound in accordance with any of the above embodiments under conditions such that the polyamino compound and the multi-arm polymer crosslink after administration.

In some embodiments, the present disclosure is directed to methods making a multi-arm polymer comprising (a) reacting a precursor multi-arm polymer that comprises at least one polymer arm having a terminal hydroxyl group with a cyclic acid anhydride compound that comprises one or more radiopaque atoms, thereby forming an intermediate multi-arm polymer that comprises a radiopaque cyclic acid anhydride residue that is linked to the at least one polymer arm through an ester group and has a free carboxylic acid group, and (b) reacting the free carboxylic acid group with a hydroxyl group of an electrophilic-group-forming compound in an ester coupling reaction.

In some embodiments, the present disclosure is directed to methods of making a multi-arm polymer comprising (a) reacting a precursor multi-arm polymer that comprises at least one polymer arm having a terminal hydroxyl group with a cyclic-ester-containing compound, thereby forming a first intermediate multi-arm polymer that comprises a hydroxyalkyl ester group that is linked to the at least one polymer arm through a first ester group, (b) reacting the hydroxyl group of the hydroxyalkyl ester group of the first intermediate multi-arm polymer with a cyclic acid anhydride compound that comprises one or more radiopaque atoms, thereby forming a second intermediate multi-arm polymer that comprises a radiopaque cyclic acid anhydride residue that is linked to the at least one polymer arm through a second ester group and has a free carboxylic acid group, and (c) reacting the free carboxylic acid group with a hydroxyl group of an electrophilic-group-forming compound in an ester coupling reaction.

In some embodiments, the cyclic-ester-containing compound is selected from β-propiolactone, γ-butyrolactone, δ-valerolactone, and ε-caprolactone.

In some embodiments, which can be used in conjunction with any of the above embodiments, the cyclic acid anhydride compound comprises one or more iodine groups. For example, the cyclic acid anhydride compound may be an iodinated phthalic anhydride compound.

In some embodiments, which can be used in conjunction with any of the above embodiments, the electrophilic-group-forming compound is N-hydroxysuccinimide.

Potential benefits associated with the present disclosure include one or more of the following: radiocontrast is maintained or improved, complexity and cost of the manufacturing process is reduced, melting point of the solid components of the hydrogel can be maintained above 40° C. (improving storage and handling), homogeneity of the final hydrogel is improved, in vivo persistence can be tailored, and cure kinetics are maintained.

The above and other aspects, embodiments, features and benefits of the present disclosure will be readily apparent from the following detailed description.

### BRIEF DESCRIPTION OF THE DRAWINGS

FIG. 1 schematically illustrates a method for forming a radiopaque, reactive multi-arm polymer, according to an aspect of the present disclosure.

FIG. 2 schematically illustrates a method for forming a radiopaque, reactive multi-arm polymer, according to another aspect of the present disclosure.

FIG. 3 schematically illustrates a method whereby a multi-arm polymer is crosslinked with a polyamino compound, according to an aspect of the present disclosure.

FIG. 4 illustrates a delivery device, in accordance with an embodiment of the present disclosure.

### DETAILED DESCRIPTION

In some aspects of the present disclosure, radiopaque crosslinked hydrogels are provided, which are crosslinked reaction products of (a) one or more radiopaque, reactive multi-arm polymers and (b) one or more polyamino compounds. Unless indicated otherwise, as used herein the prefix “poly” means two or more. Particular radiopaque, reactive multi-arm polymers and particular polyamino compounds are described below.

In some aspects of the present disclosure, multi-arm polymers are provided that comprise at least one polymer arm, and typically a plurality of polymer arms, having a reactive radiopaque end moiety. The radiopaque end moiety comprises a radiopaque dicarboxylic acid ester moiety containing one or more radiopaque atoms. One of the two ester groups of the radiopaque dicarboxylic acid ester moiety forms a link to the polymer arm of the multi-arm polymer. The other of the two ester groups of the radiopaque dicarboxylic acid ester moiety forms part of a reactive electrophilic group.

In some embodiments the one or more radiopaque atoms of the radiopaque dicarboxylic acid ester moiety may be selected from Br, I, Bi, Ba, Gd, Ta, Zn, W and Au, among others.

In some embodiments the reactive electrophilic group may be selected, for example, from an imidazole ester group, a benzotriazole ester group, or an imide ester group. In particular beneficial embodiments, the electrophilic group is an N-succinimidyl ester group.

In some embodiments, the radiopaque dicarboxylic acid ester moiety is an iodine-containing dicarboxylate ester moiety. For example, the radiopaque dicarboxylic acid ester moiety may be an iodine-containing phthalic acid ester moiety, among other possibilities.

In some embodiments, the radiopaque, reactive multi-arm polymers are formed from precursor multi-arm polymers having polymer arms that comprise one or more hydroxyl end groups. For example the precursor multi-arm polymer may comprise a core region and a plurality of polymer arms (e.g., two, three, four, five, six, seven, eight, nine, ten, eleven, twelve or more polymer arms) each having one or more terminal hydroxyl groups.

In some embodiments, in a first step, a terminal hydroxyl group of at least one polymer arm, and typically a plurality of polymer arms, is reacted in a ring-opening reaction with a radiopaque cyclic acid anhydride compound containing one or more radiopaque atoms. This reaction step forms a multi-arm polymer having at least one polymer arm that is end capped with radiopaque cyclic acid anhydride residue, that radiopaque cyclic acid anhydride residue being linked to the polymer arm through an ester group and having a free carboxylic acid end group.

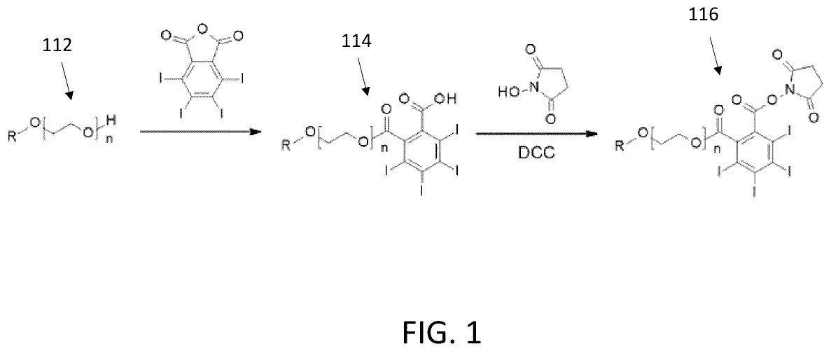

In a particular first step shown in FIG. 1, terminal hydroxyl groups of an exemplary hydroxy-terminated 8-arm PEG (112) having a core region that comprises a polyol residue R (for example, a tripentaerythritol residue core) and eight hydroxyl-terminated polyethylene oxide arms (one polymer arm is shown), where n ranges from 30 to 140 are reacted with a radiopaque cyclic acid anhydride compound, in particular, 3,4,5,6-tetraiodophthalic anhydride, also known as 4,5,6,7-tetraiodo-1,3-isobenzofuran-1,3-dione (CAS #632-80-4), in a ring-opening reaction, thereby forming an iodinated-phthalate-end-capped multi-arm PEG (114).

Other Iodinated phthalic anhydride compounds that may be employed in this reaction step include other iodinated phthalic anhydride compounds in which the aromatic ring of the phthalic anhydride compound is substituted with one or more iodine groups. Specific iodinated phthalic anhydride compounds include triiodophthalic anhydride compounds such as 4,5,7-triiodoisobenzofuran-1,3-dione,

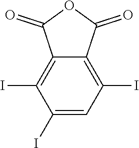 [SMILES: O=C1OC(=O)c2c(I)c(I)cc(I)c21]

and 4,5,6-triiodo isobenzofuran-1,3-dione,

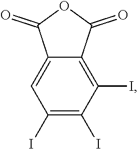 [SMILES: O=C1OC(=O)c2c1cc(I)c(I)c2I]

diiodophthalic anhydride compounds such as 4,6-diiodoisobenzofuran-1,3-dione,

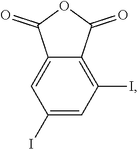 [SMILES: O=C1OC(=O)c2c(I)cc(I)cc21]

4,5-diiodoisobenzofuran-1,3-dione,

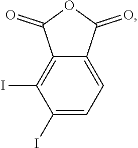 [SMILES: O=C1OC(=O)c2c1ccc(I)c2I]

5,6-diiodoisobenzofuran-1,3-dione,

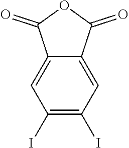 [SMILES: O=C1OC(=O)c2cc(I)c(I)cc21]

and 4,7-diiodoisobenzofuran-1,3-dione,

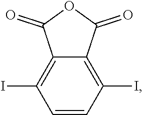 [SMILES: O=C1OC(=O)c2c(I)ccc(I)c21]

and monoiodophthalic anhydride compounds such as, 5-iodoisobenzofuran-1,3-dione,

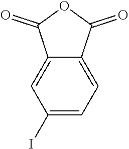 [SMILES: O=C1OC(=O)c2cc(I)ccc21]

and 4-iodoisobenzofuran-1,3-dione,

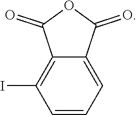 [SMILES: O=C1OC(=O)c2c(I)cccc21]

In a second step, free carboxylic acid groups of the multi-arm polymer product of the first step are reacted in an ester coupling reaction with a hydroxyl group of an electrophilic-group-forming compound, thereby producing a radiopaque multi-arm polymer having at least one polymer arm with a terminal reactive electrophilic group, more particularly, a radiopaque multi-arm polymer with at least one polymer arm having a radiopaque cyclic acid anhydride residue and a terminal reactive electrophilic group. Such a reactive electrophilic group may be selected, for example, from imidazole ester groups, benzotriazole ester groups, and imide ester groups. In certain beneficial embodiments, such an ester coupling reaction may be performed using a suitable coupling reagent, for instance, a carbodiimide coupling reagent such as dicyclohexylcarbodiimide (DCC) or diisopropylcarbodiimide (DIC).

In a particular second step shown in FIG. 1, the free carboxylic acid groups of the iodinated-phthalate-end-capped multi-arm PEG (114) from the first step are reacted in an ester coupling reaction with N-hydroxysuccinimide (CAS #6066-82-6) using DCC as a coupling reagent, to produce a radiopaque multi-arm polymer having terminal N-succinimidyl ester groups, more particularly, a radiopaque multi-arm polymer having terminal succinimidyl iodinated phthalate (SIP) groups (116).

In some embodiments, it may be desirable to introduce an additional ester group into the radiopaque, reactive multi-arm polymer that is formed. For instance, an additional ester group may be introduced in order to enhance the biodegradability of the radiopaque, reactive multi-arm polymer via hydrolysis. For example, in a first step, a terminal hydroxyl group of at least one polymer arm, and typically a plurality of polymer arms, of a precursor multi-arm polymer may be reacted in a ring-opening reaction with a cyclic-ester-containing compound, specific examples of which include lactone compounds such as β-propiolactone, γ-butyrolactone, δ-valerolactone, ε-caprolactone, and other lactone derivatives, thereby forming a first intermediate multi-arm polymer having at least one polymer arm that is end capped with a hydroxyalkyl group, the hydroxyalkyl group being linked to the polymer arm through an ester group. In other words, in the first intermediate multi-arm polymer that is formed, at least one polymer arm of which is end capped with a hydroxyalkyl ester group.

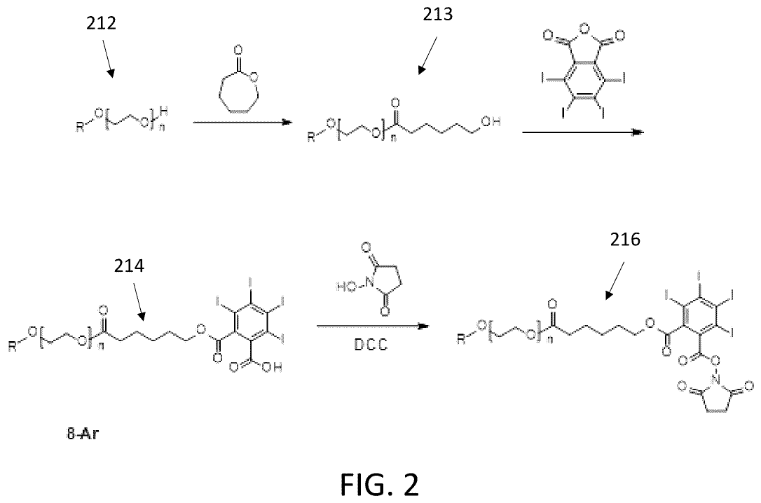

In a particular first step shown in FIG. 2, terminal hydroxyl groups of an exemplary hydroxy-terminated 8-arm PEG (212) having a core region that comprises a polyol residue R (for example, a tripentaerythritol residue core) and eight hydroxyl-terminated polyethylene oxide arms (one polymer arm is shown), where n ranges from 30 to 140, are reacted with a cyclic-ester-containing compound, in particular, ε-caprolactone, in a ring-opening reaction forming a hydroxybutyl-ester-end-capped multi-arm PEG (213).

Then, in a second step, the hydroxyl group of the at least one hydroxyalkyl group of the first intermediate multi-arm polymer is reacted in a ring-opening reaction with a radiopaque cyclic acid anhydride compound that contains one or more radiopaque atoms, thereby forming a second intermediate multi-arm polymer having at least one polymer arm that is end capped with a radiopaque cyclic acid anhydride residue, each radiopaque cyclic acid anhydride residue being linked to a polymer arm through a newly formed ester group and having a free carboxylic acid end group.

In a particular first step shown in FIG. 2, terminal hydroxyl groups of the hydroxybutyl-ester-end-capped multi-arm PEG (213) formed in the first step are reacted with a radiopaque cyclic acid anhydride compound, in particular, 3,4,5,6-tetraiodophthalic anhydride, in a ring-opening reaction forming an iodinated-phthalate-end-capped multi-arm PEG (214). Other iodinated phthalic anhydride compounds may be employed in this reaction step, including other iodinated phthalic anhydride compounds in which the aromatic ring of the phthalic anhydride compound is substituted with one or more iodine groups, such as those discussed above.

In a third step, at least one free carboxylic acid group of the second intermediate multi-arm polymer product of the second step is reacted in an ester coupling reaction with a hydroxyl group of an electrophilic-group-forming compound, thereby producing a radiopaque multi-arm polymer having at least one terminal reactive electrophilic group, more particularly, a radiopaque multi-arm polymer having at least one radiopaque cyclic acid anhydride residue and terminal reactive electrophilic group. Such a reactive electrophilic group may be selected, for example, from imidazole ester groups, benzotriazole ester groups, and imide ester groups.

In a particular third step shown in FIG. 2, the free carboxylic acid groups of the iodinated-phthalate-end-capped multi-arm PEG (214) from the second step are reacted in an ester coupling reaction with N-hydroxysuccinimide to produce a radiopaque multi-arm polymer having terminal N-succinimidyl ester groups, more particularly, a radiopaque multi-arm polymer having terminal succinimidyl iodinated phthalate groups (216). Multi-arm polymer (216) is similar to the multi-arm polymer (116) of FIG. 1, wherein an additional alky ester group (i.e., a hexanoate ester group) is provided between each polymer arm and the terminal succinimidyl iodinated phthalate group.

Although the above reaction schemes employ iodinated anhydride compounds, it should be noted that other radiopaque atoms besides iodine, including bromine, may be employed.

The above reaction schemes require a precursor multi-arm polymer having a plurality of polymer arms (e.g., having two, three, four, five, six, seven, eight, nine, ten, eleven, twelve or more arms), wherein two or more polymer arms of the precursor multi-arm polymer each comprises one or more hydroxyl end groups.

In various embodiments, the polymer arms are hydrophilic polymer arms. Such hydrophilic polymer arms may be composed of any of a variety of synthetic, natural, or hybrid synthetic-natural polymer arms. These may be selected, for example, from the following polymer arms, among others: polyether arms including poly(alkylene oxide) arms such as poly(ethylene oxide) (PEO) (also referred to as polyethylene glycol or PEG) arms, poly(propylene oxide) (PPO) arms, poly(ethylene oxide-co-propylene oxide) arms, poly(tetramethylene oxide) (PTMO) (also referred to as polytetrahydrofuran or polyTHF) arms, and poly(hexamethylene oxide) (PHMO) arms, polyoxazoline arms including poly(2-alkyl-2-oxazoline) arms such as poly(2-methyl-2-oxazoline) arms, poly(2-ethyl-2-oxazoline) arms, poly(2-propyl-2-oxazoline) arms and poly(2-isopropyl-2-oxazoline) arms, and poly(N-vinyl pyrrolidone) arms. Polymer arms for use in the multi-arm polymers of the present disclosure typically contain from 10 to 2000 monomer units, for example, ranging anywhere from 10 to 20 to 50 to 100 to 200 to 250 to 500 to 1000 to 2000 monomer units (in other words, ranging between any two of the preceding numerical values).

In various embodiments, the polymer arms extend from a core region. In certain embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, tripentaerythritol adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others. In certain beneficial embodiments, the core region comprises a residue of a polyol that contains two, three, four, five, six, seven, eight, nine, ten or more hydroxyl groups.

In certain embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T6, T8, T10 or T12 cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

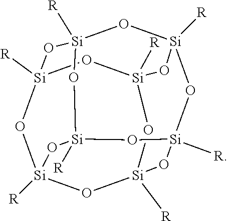

In the present disclosure, at least two R groups comprise polymer arms, and typically all R groups comprise polymer arms.

Using the above and other reaction schemes, radiopaque, reactive multi-arm polymers can be provided that have a plurality of reactive radiopaque end moieties that comprise a radiopaque dicarboxylic acid ester moiety containing one or more radiopaque atoms, one ester group of which forms a link to a polymer arm of the multi-arm polymer and the other ester group of which forms part of an electrophilic group. Moreover, in some embodiments like that shown in FIG. 2, an additional ester group, for example, an alkanoate ester group (e.g., a C₂-C₈ alkanoate ester group), can be provided between the radiopaque dicarboxylic acid ester moiety and the polymer arm.

In various aspects, the present disclosure provides a radiopaque crosslinked hydrogel that comprises a crosslinked reaction product of (a) a radiopaque, reactive multi-arm polymer like that described above and (b) a polyamino compound.

In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprise a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.). Where additional radiopacity is desired in the crosslinked hydrogel, iodinated polyamine compounds may be employed in some embodiments.

Particular examples of polyamino compounds which may be used as the multifunctional compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others.

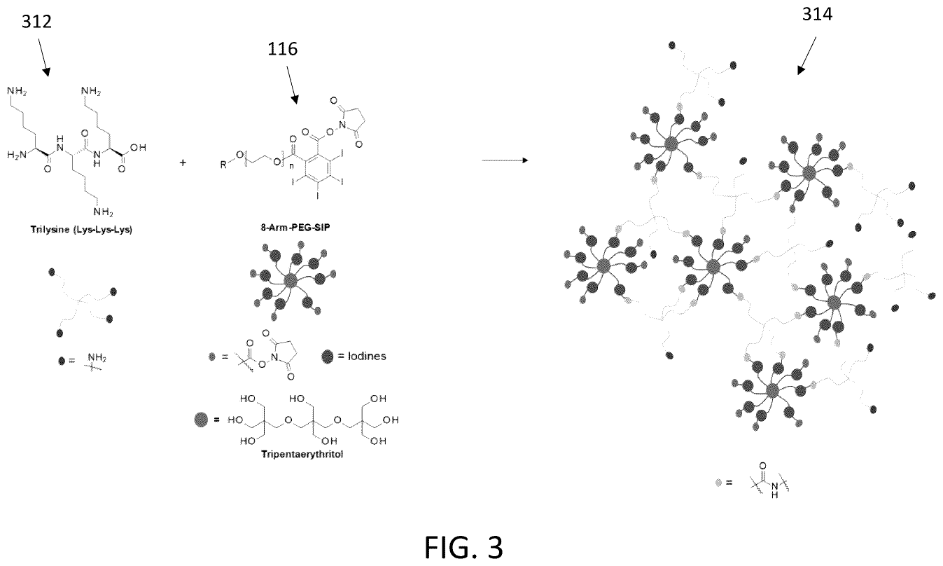

In a particular example shown schematically in FIG. 3, a radiopaque, reactive multi-arm polymer (116) like that described above, can be covalently crosslinked with a polyamino compound (312) like that described above, in particular, trilysine, which comprises primary amine groups that are reactive with the reactive groups (i.e., the N-succinimidyl ester groups) of the radiopaque, reactive multi-arm polymer (116). By reacting the polyamino compound (312) with the radiopaque, reactive multi-arm polymer (116) under basic conditions, a crosslinked product (314) is formed, which may be in the form of a crosslinked hydrogel when hydrated.

An advantage to this approach is that the iodine functionality, and thus radiopacity, is provided without sacrificing N-succinimidyl ester groups of the radiopaque, reactive multi-arm polymer. This allows reactive end groups to be provided on each of the polymer arms, thereby maximizing the crosslinking capacity of the radiopaque, reactive multi-arm polymer, without sacrificing radiopacity.

As previously noted, in various aspects of the present disclosure, a radiopaque crosslinked hydrogel is provided that comprises a crosslinked reaction product of (a) a radiopaque, reactive multi-arm polymer like that described above and (b) a polyamino compound.

In various embodiments, the radiopaque crosslinked hydrogels of the present disclosure are visible under fluoroscopy. In various embodiments, such crosslinked products have a radiopacity that is 100 Hounsfield units (HU) or more, beneficially ranging anywhere from 100 HU to 250 HU to 500 HU to 750 HU to 1000 HU to 2500 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In various embodiments, systems are provided that are configured to dispense a polyamino compound like that described above and a radiopaque, reactive multi-arm polymer like that described above under conditions such that the polyamino compound and the radiopaque, reactive polymer covalently crosslink with one another. In certain embodiments, those conditions comprise an environment having a basic pH, for example, a pH ranging from about 9 to about 11, typically ranging from about 9.5 to about 10.5, and beneficially ranging from about 9.8 to about 10.2.

In some embodiments, a system is provided that comprises (a) a first composition that comprises the polyamino compound and (b) a second composition that comprises the radiopaque, reactive multi-arm polymer. In some embodiments, a third composition in the form of an accelerant composition is provided.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including those described below.

The second composition may be a second fluid composition comprising the radiopaque, reactive multi-arm polymer or a second dry composition that comprises the radiopaque, reactive multi-arm polymer, to which a suitable fluid such as water for injection, saline, etc. can be added to form a second fluid composition. In addition to the radiopaque, reactive multi-arm polymer, the second composition may further comprise additional agents including those described below.

In some embodiments, the polyamino compound is initially combined with the radiopaque, reactive multi-arm polymer at an acidic pH at which covalent crosslinking between the reactive groups of the radiopaque, reactive multi-arm polymer and the primary amine groups of the polyamino compound is suppressed. Then, when covalent crosslinking is desired, a pH of the mixture of the polyamino compound and the radiopaque, reactive multi-arm polymer is changed from an acidic pH to a basic pH, leading to covalent crosslinking between same.

In particular embodiments, the system comprises (a) a first precursor composition that comprises a polyamino compound as described hereinabove, (b) a second precursor composition that comprises a radiopaque, reactive multi-arm polymer as described hereinabove, and (c) a third composition, specifically, an accelerant composition, that contains an accelerant that is configured to accelerate crosslinking reaction between the polyamino compound and the radiopaque, reactive multi-arm polymer.

The first precursor composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 5, typically ranging from about 3.5 to about 4.5, and more typically ranging from about 3.8 to about 4.2. In addition to the polyamino compound, the first precursor composition may further comprise additional agents, including those described below.

The second precursor composition may be a second fluid composition comprising the radiopaque, reactive multi-arm polymer or a second dry composition that comprises the radiopaque, reactive multi-arm polymer from which a second fluid composition is formed, for example, by the addition of a suitable fluid such as water for injection, saline, or by the addition of the first fluid composition comprising the polyamino compound that is buffered to an acidic pH. In addition to the radiopaque, reactive multi-arm polymer, the second precursor composition may further comprise additional agents, including those described below.

In a particularly beneficial embodiment, the first precursor composition is a first fluid composition comprising the polyamino compound that is buffered to an acidic pH and the second precursor composition comprises a dry composition that comprises the radiopaque, reactive multi-arm polymer. The first precursor composition may then be mixed with the second precursor composition to provide a prepared fluid composition that is buffered to an acidic pH and comprises the polyamino compound and the radiopaque, reactive multi-arm polymer. In a particular example, a syringe may be provided that contains a first fluid composition comprising the polyamino compound that is buffered to an acidic pH, and a vial may be provided that comprises a dry composition (e.g., a powder) that comprises the radiopaque, reactive multi-arm polymer. The syringe may then be used to inject the first fluid composition into the vial containing the radiopaque, reactive multi-arm polymer to form a prepared fluid composition that contains the polyamino compound and the radiopaque, reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, including those described below. In a particular example, a syringe may be provided that contains the fluid accelerant.

A prepared fluid composition that is buffered to an acidic pH and comprises the polyamino compound and the radiopaque, reactive multi-arm polymer as described above, and a fluid accelerant composition that is buffered to basic pH as described above, may be combined form crosslinked hydrogels, either in vivo or ex vivo.

Examples of additional agents for use in the above-described compositions include therapeutic agents. Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists among others.

Examples of additional agents also include colorants such as brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include imaging agents in addition to the iodine present in the radiopaque products. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, a system is provided that include one or more delivery devices for delivering first and second compositions to a subject.

In some embodiments, a delivery device is provided that includes (a) a first reservoir that contains a first fluid composition that comprises a polyamino compound as described above and (b) a second reservoir that contains a second fluid composition that comprises a radiopaque, reactive multi-arm polymer as described above.

In some embodiments, a delivery device is provided that includes (a) a first reservoir that contains a first fluid composition that comprises a polyamino compound as described above and a radiopaque, reactive multi-arm polymer as described above, wherein the first fluid composition is buffered to an acidic pH to inhibit covalent crosslinking, such as the prepared fluid composition previously described and (b) a second reservoir that contains a second fluid composition, such as the fluid accelerant composition described above.

In each of the above cases, during operation, the first composition and second composition are dispensed from the first and second reservoirs and combined, whereupon the polyamino compound and the radiopaque, reactive multi-arm polymer and crosslink with one another to form a hydrogel.

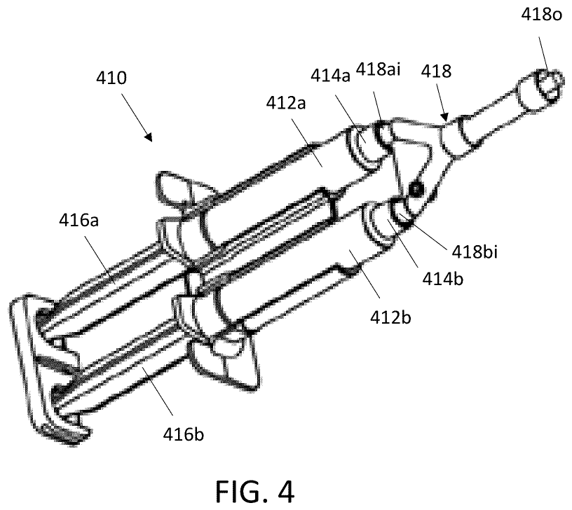

Regardless of the first and second compositions selected, in particular embodiments, and with reference to FIG. 4, the system may include a delivery device 410 that comprises a double-barrel syringe, which includes first barrel 412a having a first barrel outlet 414a, which first barrel contains the first composition, a first plunger 416a that is movable in the first barrel 412a, a second barrel 412b having a second barrel outlet 414b, which second barrel 412b contains the second composition, and a second plunger 416b that is movable in the second barrel 412b. In some embodiments, the device 410 may further comprise a mixing section 418 having a first mixing section inlet 418ai in fluid communication with the first barrel outlet 414a, a second mixing section inlet 418bi in fluid communication with the second barrel outlet, and a mixing section outlet 4180.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments. As another example, although iodine groups are described above, other radiopaque halogen groups including bromine may be substituted. The invention's scope is, of course, defined in the language in which the appended claims are expressed.

1. A multi-arm polymer comprising at least one polymer arm that comprises a reactive radiopaque end moiety, the radiopaque end moiety comprising a radiopaque dicarboxylic acid ester moiety containing one or more radiopaque atoms, wherein one of two ester groups of the radiopaque dicarboxylic acid ester moiety forms a link to the polymer arm, and wherein the other of the two ester groups of the radiopaque dicarboxylic acid ester moiety corresponds to a portion of a reactive electrophilic group.

2. The multi-arm polymer of claim 1, comprising at least two polymer arms that comprise a reactive radiopaque end moiety.

3. The multi-arm polymer of claim 1, wherein the reactive electrophilic group is selected from an imidazole ester group, a benzotriazole ester group, and an imide ester group.

4. The multi-arm polymer of claim 1, wherein the one or more radiopaque atoms of the radiopaque dicarboxylic acid ester moiety are selected from Br, I, Bi, Ba, Gd, Ta, Zn, W and Au.

5. The multi-arm polymer of claim 1, wherein the radiopaque dicarboxylic acid ester moiety is an iodine-containing dicarboxylate ester moiety.

6. The multi-arm polymer of claim 1, further comprising an additional ester group disposed between the radiopaque dicarboxylic acid ester moiety and the at least one polymer arm.

7. The multi-arm polymer of claim 6, wherein the additional ester group is a C₂-C₈ alkanoate ester group.

8. The multi-arm polymer of claim 1, wherein the at least one polymer arm is selected from poly(alkylene oxide) arms, polyoxazoline arms, and poly(N-vinyl pyrrolidone) arms.

9. The multi-arm polymer of claim 1, wherein the at least one polymer arm extends from a core selected from a polyol residue core and a silsesquioxane core.

10. A system for forming a hydrogel that comprises: the multi-arm polymer of claim 1 and a polyamino compound comprising a plurality of primary amine groups that form crosslinks with the multi-arm polymer.

11. The system of claim 10, wherein the polyamino compound comprises alkylamino groups that include the plurality of primary amine groups.

12. The system of claim 11, wherein the alkylamino groups are of the formula —(CH₂)ₓ—NH₂, where x is 1, 2, 3, 4, 5 or 6.

13. The system of claim 10, wherein the system comprises a first precursor composition that comprises the polyamino compound, a second precursor composition that comprises the multi-arm polymer, and an optional accelerant composition.

14. The system of claim 13, wherein the first precursor composition is provided in a syringe barrel, the second precursor composition is provided in a vial, and the accelerant composition is provided in a syringe barrel.

15. A medical hydrogel formed by crosslinking the multi-arm polymer of claim 1 and a polyamino compound comprising a plurality of primary amine groups that form crosslinks with the multi-arm polymer.

16. A method of treatment comprising administering to a subject a mixture that comprises the multi-arm polymer of claim 1 and a polyamino compound comprising a plurality of primary amine groups that form crosslinks with the multi-arm polymer under conditions such that the polyamino compound and the multi-arm polymer crosslink after administration.

17. A method of making a multi-arm polymer comprising (a) reacting a precursor multi-arm polymer that comprises at least one polymer arm having a terminal hydroxyl group with a cyclic acid anhydride compound that comprises one or more radiopaque atoms, thereby forming an intermediate multi-arm polymer that comprises a radiopaque cyclic acid anhydride residue that is linked to the at least one polymer arm through an ester group and has a free carboxylic acid group, and (b) reacting the free carboxylic acid group with a hydroxyl group of an electrophilic-group-forming compound in an ester coupling reaction.

18. The method of claim 17, where the cyclic acid anhydride compound comprises one or more iodine groups.

19. A method of making a multi-arm polymer comprising (a) reacting a precursor multi-arm polymer that comprises at least one polymer arm having a terminal hydroxyl group with a cyclic-ester-containing compound thereby forming a first intermediate multi-arm polymer that comprises hydroxyalkyl ester group that is linked to the at least one polymer arm through a first ester group, (b) reacting the hydroxyl group of the hydroxyalkyl ester group of the first intermediate multi-arm polymer with a cyclic acid anhydride compound that comprises one or more radiopaque atoms thereby forming a second intermediate multi-arm polymer that comprises a radiopaque cyclic acid anhydride residue that is linked to the at least one polymer arm through a second ester group and has a free carboxylic acid group, and (c) reacting the free carboxylic acid group with a hydroxyl group of an electrophilic-group-forming compound in an ester coupling reaction.

20. The method of claim 19, wherein the cyclic-ester-containing compound is selected from β-propiolactone, γ-butyrolactone, δ-valerolactone, and ε-caprolactone.
